(19) United States
(12) Patent Application Publication
(10) Pub. No.: US 2006/0115523 A1
(43) Pub. Date: Jun. 1, 2006

(54) Sterically stabilized liposome and triamcinolone composition for treating the respiratory tract of a mammal

(76) Inventors: Kameswari S. Konduri, Irving, TX (US); Sandhya Nandedkar, Brookfield, WI (US); Nejat Duzgunes, Mill Valley, CA (US); Pattisapu Ram Jogi Gangadharam, Irving, TX (US); Jogi Pattisapu, Orlando, FL (US)

Correspondence Address: F. Lindsey Scott, Suite B, 2329 Coit Road, Plano, TX, 75075, US

(21) Appl. No.: 11/287,703

(22) Filed: Nov. 22, 2005

Related U.S. Application Data
(60) Provisional application No. 60/632,181, filed on Dec. 1, 2004.

(51) Int. Cl. A61K 9/127

(52) U.S. Cl. 424/450; 977/907

(57) ABSTRACT

This invention relates to a composition containing a sterically stabilized liposome and triamcinolone, effective for the treatment of a mammal, with the composition being adapted for administration as an aerosol and with the composition providing effective treatment for a period of time at least 1.5 times as long as the effective time for treatment with triamcinolone alone. This invention also relates to a method for treating a mammal respiratory tract with the composition.

### RELATED APPLICATION

This application is entitled to and hereby claims the benefit of the filing date of provisional application No. 60/632,181 filed Dec. 1, 2004 by Kameswari Surya Konduri, et al.

### FIELD OF THE INVENTION

This invention is directed to a sterically stabilized liposome and triamcinolone composition effective for the aerosol delivery of the composition which is effective in the treatment of the respiratory tract of a mammal The composition provides effective treatment for a period of time at least 1.5 times as long as the effective time for aerosol treatment of the mammal with a comparable quantity of triamcinolone alone.

### BACKGROUND OF THE INVENTION

Asthma is a common disease that causes recurrent symptoms, repeated hospitalizations and an increased risk of sudden death. It is the most common childhood illness and affects five to ten percent of the population in North America. Asthma also accounts for the most hospitalizations of pediatric age people, the most missed school days and the most missed workdays at an estimated cost of $6.2 billion in 1988.

Asthma is characterized by acute bronchial restriction, chronic lung inflammation and airway hypersensitivity which results in chronic inflammation and airway remodeling that leads to progressive and possibly irreversible airway damage. The most effective therapy focuses on the early stages of the disease before the vicious cycle of inflammatory changes can become irreparable. The disease usually starts in early childhood and most commonly before five years of age. Thus, appropriate management of asthma in childhood may have a greater impact on the course of the disease than interventions later in life.

Asthma is primarily an inflammatory disease that can be prevented, though not cured. The inflammation occurs after a triggering agent (allergen) induces the release of histamine from the mast cells. Histamine and other mediators released from the mast cells attract numerous inflammatory cells (i.e., lymphocytes, eosinophils) to the bronchial epithelium along with their pro-inflammatory cytokines and mediators. IL-4 is an important cytokine. It plays a major role in differentiation of CD4 T lymphocytes into pro-inflammatory cells. i.e., TH2 subtype. Regular anti-inflammatory medication use is crucial in preventing airway remodeling and irreversible lung damage that occurs in asthma.

The mainstay of asthma treatment therapy is the use of anti-inflammatory drugs (i.e., inhaled corticosteroids). As a first line therapy for patients above five years of age, inhaled corticosteroids are usually given via a metered dose inhaler twice a day. Patients under five years of age are frequently given chromoline sodium three to four times a day via a nebulizer. A nebulizer form of Budesonide (BUD), which is a potent inhaled corticosteroid, given twice a day is being used as a first line therapy in patients under five years.

Although current inhaled corticosteroids are very effective in preventing the massive inflammation that occurs with asthma, they do have some major drawbacks. One is that these drugs must be given at least daily to be effective. This daily dosage requirement may lead to non-adherence by the patient. Since adherence to daily use of inhaled corticosteriods by the patient is critical in interrupting the chronic inflammation that occurs in asthma, this becomes a focal issue for effective therapy. Further the effective use of a metered dose inhaler is very technique-dependent. Typically only about three to eight percent of a given dose is delivered to the lungs via a metered dose inhaler. In addition, only a fraction of the drug reaches the lower or peripheral airways. Additionally the inhaled corticosteroids have a short half-life in the body and have potential toxicity when used in higher doses. These are serious disadvantages to the use of corticosteroid drugs in conventional therapy.

In an abstract published by the present inventors in the Journal of Allergy Clinical Immunology entitled “Efficacy of Liposome Encapsulated Budesonide in Experimental Asthma,” February, 2001, Vol. 107, No. 2, it is disclosed that BUD encapsulated in sterically stabilized liposomes prevents asthma inflammation in lower doses given at less frequent intervals. Test results are summarized demonstrating an improvement with budesonide. The abstract does not disclose a suitable sterically stabilized liposome, suitable types of sterically stabilized liposomes, or any method for producing a suitable sterically stabilized liposome or the use of drugs other than BUD.

In “Efficacy of Liposomal Budesonide in Experimental Asthma,” Kondari, Kameswari S., M. D., Nandedkar, Sandhya, M. D., Duzgunes, Nejat, Ph.D., Suzara, Vincent, Arlwohl, James, C. V. M., Bunte, Ralph, D. V. M. and Pattisapu, R. J. Sangadharen, Gangadbaram, Ph.D., Journal of Allergy Clinical Immunology, February 2003, tests were reported showing that with a specific disclosed stabilized liposome with budesonide extended asthma relief of allergic inflammation is achieved in mice. There is no showing that other drugs would be similarly useful. The article discloses that incorporation of budesonide into conventional liposomes results in rapid redistribution of the budesonide by leaking from the liposomes into the medium. Triamcinolone alone is not suitable for use in a nebulizer because even the more common form, triamcinolone acetonide, is a sticky solid (powder) and difficult to use as a powdered inhalant and in a nebulized form. Triamcinolone, if used as an aerosol requires combination with a propellant.

In “The Effect of Budesonide Encapsulated in Liposomes on Airway Hyperresponsiveness,” Sandhya D. Nandedkar, Kameswari S. Konduri, David A. Rickaby and Nejat Duzgunes, Medical College of Wisconsin and Zablocki, Va. Medical Center, Milwaukee, Wis., University of the Pacific School of Dentistry, San Francisco, Calif., an abstract presented at the National Allergy Meeting, March, 2003 in Denver, Colo., tests are reported using budesonide and stabilized liposomes to reduce airway hyperresponsiveness and pulmonary inflammation for extended periods of time as follows:
“We have previously shown that budesonide encapsulated in liposomes given once a week decreased lung inflammation equivalent to daily budesonide therapy in experimental asthma. Liposome composition is similar to lung surfactant and, therefore, may aid in decreasing airway hyperresponsiveness (AHR). However, it is not clearly defined whether inhaled glucocorticosteroids have any effect on AHR. We hypothesized that weekly administration of budesonide encapsulated in liposomes would decrease AHR to methacholine in ovalbumin-sensitized C57B1/6 mice. AHR to methacholine was measured in spontaneously breathing, tracheally intubated mice that received increasing doses of methacholine (up to 3 mg) intraperitoneally. The sensitized mice were divided into four groups and received for 4 weeks: (1) nebulized budesonide daily (2) nebulized budesonide encapsulated in liposomes weekly (3) nebulized budesonide weekly (4) no budesonide treatment. Normal mice were maintained as additional controls. Only the budesonide encapsulated in liposomes, administered weekly, significantly decreased AHR to methacholine (p<0.05) and was comparable to normal mice. These data suggest that budesonide encapsulated in liposomes, administered weekly, can decrease lung inflammation and AHR. Daily budesonide, however, was noted to only decrease lung inflammation and did not provide any benefit in decreasing AHR to methacholine. This is the first study to show the efficacy of budesonide encapsulated in liposomes as a treatment that can be given weekly, to decrease AHR to methacholine. This new treatment modality allows a method for using very low doses and less frequent dosing intervals to decrease both lung inflammation and AHR.”

This abstract does not show that other drugs would be similarly effective to achieve the extended effectiveness.

In view of the likelihood of detrimental effects based upon the use of the corticosteroids and the frequency with which the corticosteroids and other drugs are required, a continued effort has been directed to the development of improved techniques for administering a drug to the respiratory tract of a mammal so that it may be administered more effectively and so that the effectiveness of the drug can be achieved using smaller doses.

### SUMMARY OF THE INVENTION

The invention comprises a composition comprising a sterically stabilized liposome carrier in combination with triamcinolone, the composition being adapted for aerosol administration to a mammal, compatible with the respiratory tract of a mammal and effective to extend the effective life of triamcinolone in the respiratory tract by a time equal to at least twice the effective life of triamcinolone alone.

The invention also comprises a method for treating the respiratory tract of a mammal by administering an effective amount of a composition as an aerosol comprising a sterically stabilized liposome in combination with triamcinolone, the composition being compatible with the respiratory tract of a mammal and effective to extend the effective life of triamcinolone in the respiratory tract of the mammal by a time equal to at least twice the effective life of triamcinolone alone.

### BRIEF DESCRIPTION OF THE DRAWINGS

FIG. 1 shows histopathology test results;

FIG. 2 shows eosinophil peroxidase (EPO) test results;

FIG. 3 shows serum IgE levels;

FIG. 4 shows airway hyperresponsiveness (AHR) to methacholine challenge test results; and,

FIG. 5 shows lung tissues from the experimental groups.

### DESCRIPTION OF PREFERRED EMBODIMENTS

Liposomes are well known materials that constitute primarily phospholipid bilayer vesicles of many types that can encapsulate a variety of drugs and are avidly phagocytosed by macrophages in the body. The various interactions of the liposomes can be generalized into four categories: (1) exchange of materials, primarily lipids and proteins with cell membranes; (2) absorption or binding of liposomes to cells; (3) cell internalization of liposomes by endocytosis or phagocytosis once bound to the cell; and, (4) fusion of bound liposomes with the cell membrane. In all these interactions, there is a strong dependence on lipid composition, type of cell, presence of specific receptors and many other parameters.

Liposomes have been used to provide drugs in mammal bodies, particularly when it is desired to apply the drugs to specific areas for specific applications. Liposomes have been used to encapsulate antibiotics, antiviral agents and the like and have been shown to enable enhanced efficacy against a variety of infectious diseases. A major drawback of liposomes is that they have a relatively short life in a mammal body. Most applications have used liposomes in the bloodstream.

Liposomes are small spherical structures that contain a polar surface and nonpolar interior, similar to the cell membrane. Conventional liposomes are characterized by a nonspecific reactivity with the milieu, whereas the sterically stabilized liposomes are relatively inert, due to their surface coating with poly(ethylene) glycol (PEG) and therefore are less reactive or non-reactive to the environment. Studies using conventional liposomes containing either triamcinolone or hydrocortisone have shown that the steroids redistribute and leak rapidly from the liposome into the medium secondary to the partition coefficient. To date there are no reported studies using sterically stabilized liposomes to encapsulate triamcinolone.

To extend the life of liposomes in a mammal body, attempts have been made to develop sterically stabilized liposomes, which have a longer life in a mammal body. Attempts to extend the life of liposomes have included the use of poly(ethylene glycol), natural glycolipids, surfactants, polyvinyl alcohol, polylactic acid, polyglycolic acid, polyvinyl pyrrolidine, polyacrylamine and other materials in various combinations with the liposomes in attempts to provide sterically stabilized liposomes which are effective for drug delivery and which are compatible with a mammal circulatory system. A wide variety of such sterically stabilized liposomes have been developed for a wide variety of drug deliveries for a wide variety of specific mammal disorders. The most prominent sterically stabilized liposomes utilize distearoylphosphatidylcholine or hydrogenated soy phosphatidylcholine as the primary phospholipid.

Sterically stabilized liposomes have enhanced stability and decreased immunogencity due to their surface coating with PEG. PEG derivatives can be prepared and purified inexpensively and many have already been approved for pharmaceutical use, such as PEG adenine deaminase (ADA) and liposomes containing these derivatives make them ideal for therapeutic application. In addition, it has been reported that empty liposomes (without drug) can decrease inflammation which may be an additional benefit of using liposomes as a delivery system.

For use in the present invention, it has been necessary to produce sterically stabilized liposomes which are compatible with a mammal respiratory system and lungs, adapted for aerosol, especially nebulizer, administration to the mammal and which have an extended life in the lungs and respiratory tract. For instance, the most commonly used sterically stabilized liposome uses distearoylphosphatidylcholine as the primary phospholipid. Due to its very high phase transition temperature, this lipid is not considered compatible with lung surfactant, which may contain dipalmitoyl lipids with shorter acyl chains and a lower phase transition temperature.

The sterically stabilized liposomes of the present invention have a composition such that they are readily administered to the mammal as an aerosol and will remain stable in the presence of serum and in the extra-cellular environment. They preferentially localize to the lung when delivered intravenously, especially to areas of inflammation as commonly seen in asthma. These sterically stabilized liposomes are amenable to nebulization. The combination of these sterically stabilized liposomes with triamcinolone in the treatment of mammalian respiratory tract diseases has been shown herein for the treatment of lung inflammation and airway hyperresponsiveness.

Alternatively, the sterically stabilized liposomes may also include significant quantities, up to 90%, of head groups comprising phosphatidylglycerol. This mixed material, ideally at lower phosphatidylglycerol mole fractions than phosphatodylcholine, is considered to be somewhat more compatible with lung fluids than is phosphatidylcholine alone. For certain drugs, rendering the liposomes pH-sensitive may increase the efficacy of the drug, since it may facilitate the rapid breakdown of internalized liposomes at the low pH found in the endocytotic pathway, for example in alveolar macrophages or in Type II cells, as well as the destabilization of the membrane of the endocytotic vesicles and endosomes. Such pH-sensitivity may be achieved by the inclusion of a lipid moiety that is negatively charged at neutral pH, but becomes protonated at pH 6.5 and lower. Such lipids include cholesteryl hemisuccinate, diacyl succinylglycerol, oleic acid, or the like. These protonatable lipids are usually incorporated into the liposome membrane together with a phosphatidylethanolamine with unsaturated double bonds in the acyl chain, such as dioleoylphophatidylethanolamine.

A further component of the sterically stabilized liposomes may be poly(ethylene glycol), in the molecular range from about 500 to about 5,000 daltons. This component is normally covalently linked to a phosphatidylethanolamine moiety and is included as such during the formulation of the liposome. The covalent bond can be designed such that it will be cleaved under physiological conditions, such as reducing conditions or low pH in endocytotic vesicles. The poly(ethylene glycol) may also be linked to a fatty acid to facilitate its anchoring to the liposome. In either case it is possible to insert the poly(ethylene glycol)-containing lipid into the drug-containing liposome after the latter has been formed. This enables some versatility in the preparation of the liposome, and also gives the option of having the poly(ethylene glycol) only on the outside of the liposome.

The effect of the sterically stabilized liposomes in combination with triamcinolone is more pronounced than currently available drug therapies. As demonstrated more thoroughly in the following examples, this stability may allow triamcinolone to be administered, in combination with the sterically stabilized liposomes via a nebulizer treatment, only once every one to two weeks. The dosage used in these treatments is typically the same or similar to that used on a daily basis and is therefore safe. The effective life of triamcinolone administered in combination with the sterically stabilized liposomes in the respiratory tract has thus been extended up to at least seven times the effective life of triamcinolone alone. Sustained action of triamcinolone has been obtained at much lower dosages with a reduction in toxicity risk and in cost. No suggestion in the prior art is known that extended life could be obtained with these sterically stabilized liposomes for aerosol triamcinolone treatments for asthma, particularly for lung inflammation and airway hyper-responsiveness using sterically stabilized liposomes adapted for use in the lungs and airway. The inhaled steroid alone is less effective, especially with respect to airway hyperresponsiveness, than the triamcinolone in combination with the sterically stabilized liposomes of the present invention.

The sterically stabilized liposomes of the present invention comprise sterically stabilized liposomes that are compatible with the respiratory tract of a mammal and which are effective to extend the effective life of triamcinolone in the respiratory tract by a time equal to at least twice the effective life of triamcinolone alone. The sterically stabilized liposomes of the present invention are tailored to be compatible with naturally occurring fluids found in the lungs. The sterically stabilized liposomes are also tailored to accommodate the surfactant nature of some of the fluids found in the lungs so that the sterically stabilized liposomes of the present invention provide long stability in the lungs and when used to encapsulate or combine with triamcinolone have been found to be effective to extend the effective life of triamcinolone administered using the sterically stabilized liposome carriers of the present invention.

The sterically stabilized liposomes of the present invention comprise phosphatidylcholine. These materials may be synthetically derived or they may be derived from chicken eggs or soybeans. If derived from eggs they contain acyl groups having varying numbers of carbon atoms, dependent upon the variety and diet of the chicken that produces the eggs. The phosphatidylcholine is typically present in a relatively significant quantity in the sterically stabilized liposomes and may comprise the only head group for the sterically stabilized liposomes.

Desirably, the sterically stabilized liposomes may be tailored to the particular mammalian lung system contemplated. It is considered, however, that such sterically stabilized liposomes will fall within the criteria defined above for the liposomes.

Further the sterically stabilized liposomes may comprise at least one of phosphatidylcholine, phosphatidylglycerol, and poly(ethylene glycol)-distearyolphosphatidyldiethanolamine, lipid conjugated polyoxyethylene, lipid conjugated polysorbate, or lipids conjugated to other hydrophilic steric coating molecules safe for in vivo use.

Particularly preferred material is phosphatidylcholine, phosphatidylglycerol, poly(ethylene glycol)-distearyolphosphatidyldiethanolamine. This sterically stabilized liposome was used in the test shown in the Example.

Any of the head groups or the poly(ethylene glycol), may be attached to acyl groups containing from about 8 to about 22 and desirably about 8 to about 18 carbon atoms. Preferably, from about 16 to about 18 carbon atoms are present in the acyl groups. Such groups comprise distearoyl, stearoyl oleoyl, stearoyl palmitoyl, dipalmitoyl, dioleoyl, palmitoyl oleoyl, dipalmitoleoyl and the like. If shorter chains are used, such as dipalmitoyl, palmitoyl, dimyristoyl, didodecanoyl, didecanoyl or dioctanoyl, the poly(ethylene glycol)-lipid is likely to exchange into biological milieu. This may in some instances permit the liposome to better partition onto lung surfactant after shedding or exchanging its poly(ethylene glycol) moiety.

A particularly preferred material is phosphatidylcholine, phosphatidylglycerol, poly(ethylene glycol)-distearyolphosphatidyldiethanolamine. This sterically stabilized liposome was used in the tests shown in the Example.

Triamcinolone has been shown herein to provide surprising desirable results when used in combination with the sterically stabilized liposomes discussed above. The combination is considered to be a novel and effective extended treatment system for triamcinolone with the potential to reduce toxicity and improve patient compliance. This combination also provides a triamcinolone composition which can be administered by a nebulizer.

The combined sterically stabilized liposomes and triamcinolone form unilamellar or multilamellar vesicles of sizes from about 0.05 to about 10 micrometers. Preferably the composition is prepared to have substantially homogeneous sizes in a selected size range, with the average diameter typically being from about 0.05 to about 0.8 micrometers. One method for obtaining the desired size is extrusion of the composition through polycarbonate membranes having pores of a selected size, such as from about 0.05 to about 2 micrometers.

Most previously disclosed sterically stabilized liposomes have been used in attempts to extend the effective life of drugs used in the bloodstream of mammals. These sterically stabilized liposomes must exist in a radically different environment than in the respiratory tract of a mammal. Particularly in the lungs, certain surfactant requirements exist for materials that are compatible with the fluids in the lungs and the like. Further the sterically stabilized liposomes delivered to the lungs are not as susceptible to attack by phagocytotic cells as are sterically stabilized liposomes used to position drugs in the bloodstream, which are eventually cleared mostly by liver and spleen macrophages. Further most uses of sterically stabilized liposomes in combination with drugs in the bloodstream are administered via intravenous injections. While it is not clear what mechanisms exist that permit sterically stabilized liposomes to exist for longer periods of time in certain portions of the body than would be anticipated for liposomes that were not sterically stabilized, it is clear that the sterically stabilized liposomes of the present invention are remarkably stable in the respiratory tract environment and are effective to greatly extend the effective life of triamcinolone as used herein to treat various ailments of the respiratory tract.

The preparation of the sterically stabilized liposomes, the combination of the triamcinolone with the sterically stabilized liposomes, and treatments of mice according to the present invention, are demonstrated in the following examples. Studies are presented to define optimum doses, frequency of dosing intervals to decrease lung inflammation, airway responsiveness to methacholine challenge, as well as toxicity of frequent dosing of the triamcinolone with a sterically stabilized liposome complex.

The hypothesis was tested in a mouse model of asthma. The Example shows the optimal doses and frequency of dosing intervals to decrease lung inflammation, airway responsiveness to methacholine challenge, as well as toxicity of frequently dosing the drug-liposome complex. Studies were also performed to evaluate the stability of the drug-liposome complex. Liposomes are lipid bilayer vesicles, can be sterically stabilized with polyethylene glycol-conjugated lipids and were prepared so as to encapsulate or incorporate the steroid. Then the drug-containing liposome preparation was tested in a mouse/asthma model. The mouse/asthma model was produced in C57B1/6 mice using ovalbumin (OVA) sensitization. Experiments were conducted on day 25 after sensitization was completed (baseline). The sensitized animals received aerosolized: 1) 20 μg of triamcinolone encapsulated in sterically stabilized liposomes weekly, 2) 40 μg triamcinolone encapsulated in sterically stabilized liposomes weekly, 3) empty sterically stabilized liposomes (without drug) weekly. All treatment groups were compared to untreated sensitized and unsensitized normal mice. Histopathological examination of the lung tissues and serial measurements of eosinophil-peroxidase activity, peripheral blood eosinophil counts, total serum IgE levels were obtained weekly and airway responsiveness to methacholine challenge on spontaneously breathing, intubated conscious mice was obtained every week for four weeks.

The results showed that both 20 μg and 40 μg doses of triamcinolone significantly decreased lung inflammation on histopathological evaluation, peripheral blood eosinophils, along with airway hyperresponsiveness to methacholine challenge. Serum IgE levels were significantly decreased with the 40 μg dose.

### EXAMPLE

Methods

Ovalbumin Sensitization of C57B1/6 Mice

Six to eight week old male C57B1/6 mice were sensitized with ovalbumin after one-week acclimatization and quarantine in the animal house. The animals were provided ovalbumin-free diet and water ad libitum and were housed in an environmentally controlled, pathogen-free animal facility. All animal protocols were approved by the Animal Care Committee of the Medical College of Wisconsin and were in agreement with the National Institute of Health's guidelines for the care and use of laboratory animals.

The animals were sensitized with ovalbumin (OVA). On day 0, the mice underwent subcutaneous ovalbumin implantation as follows: the mice were anaesthetized with methoxyflurane given by inhalation. A small surgical incision (approximately 0.5 cm) was made on the dorsal aspect in the cervical region. The cutaneous and subcutaneous layers were separated and a fragmented heat coagulated OVA implant was inserted. The skin and layers were closed using sterile staples.

Starting on the fourteenth day after the subcutaneous OVA implantation, the mice were given a 30-minute aerosolization of 6% OVA solution on alternate days for a period of 10 days (days 14-24). This method of sensitization led to significant elevations in eosinophil peroxidase (EPO) activity, peripheral blood eosinophil count (EOS), and serum IgE levels along with lung inflammation on histopathological examination by day 24, in our preliminary studies (19, 20).

Drugs and Reagents

Triamcinolone for encapsulation and N-2-hydroxethylpiperazine-N′-2-ethanesulfonic acid (HEPES) was purchased from Sigma Chemical Co. (St. Louis, Mo.). Phosphatidylcholine (PC), phosphatidylglycerol (PG), and poly(ethylene glycol) (PEG)-distearoylphosphatidylethanolamine (DSPE) were obtained from Avanti Polar Lipids (Alabaster, Ala.). Cholesterol (chol) was purchased from Calbiochem (La Jolla, Calif.) and NaCl and KCl from Fisher Scientific, (Pittsburgh, Pa.). Methacholine was purchased from Sigma Chemicals (St. Louis, Mo.).

Liposome Preparation

Triamcinolone was encapsulated into (PG-PC-PEG-DSPE-chol). The lipids were mixed in chloroform. Triamcinolone was dissolved first in chloroform:methanol, 2:1, and added to the lipid mixture. Lipids and drug were dried onto the sides of a round-bottom glass flask or glass tube by rotary evaporation. The dried film was then hydrated by adding sterile 140 mM NaCl, 10 mM HEPES (pH 7.4) and vortexing. The resulting multilamellar liposome preparation was extruded 21 times through polycarbonate membranes (either 0.2 or 0.8 μm pore-diameter; Nuclepore, Pleasanton, Calif.) using an Avestin (Toronto, Canada) extrusion apparatus.

Bronchioalveolar Lavage (BAL) Fluid Isolation

The animals were sacrificed by an overdose of methoxyflurane given by inhalation. The trachea was exposed and cannulated with a ball-tipped 24-gauge needle. The lungs were lavaged three times with 1 ml PBS. All the washings were pooled and the samples frozen at −70° C. The samples were later thawed and assayed for determining EPO activity and cytokines.

Histopathology Observations

Histopathological examination was performed on lungs fixed with 10% formalin in PBS. Tissue samples were obtained from the trachea, bronchi, large and small bronchioles, interstitium, alveoli, and pulmonary blood vessels. The tissues were embedded in paraffin, sectioned at 5 μm thickness and then stained with hemotoxylin and eosin and periodic acid Schiff (PAS) stain, and analyzed using light microscopy. Coded slides were examined in a blinded fashion, to evaluate the effect of therapy on lung inflammation. Objective measurements of histopathological changes include number of eosinophils surrounding the bronchi, aggregation of eosinophils around blood vessels (perivascular), accumulation of other inflammatory cells, presence of desquamation and hyperplasia of the airway epithelium, mucus formation in the lumen of the airways and infiltration of inflammatory cells surrounding the alveoli.

QUANTATIVE HISTOPATHOLOGY SCORING SYSTEM

| | | LARGE | SMALL | ALVEOLAR | |
|---|---|---|---|---|---|
| TRACHEA | BRONCHI | BRONCHIOLES | BRONCHIOLES | INTERSTITIUM | Alveoli |
| Epithelium | Epithelium | Epithelium | Epithelium | Thickening(mm) | Thickening(mm) |
| Hyperplasia(mm) | Hyperplasia(mm) | Hyperplasia(mm) | Hyperplasia(mm) | Edema(mm) | Edema(mm) |
| Desquamation | Desquamation | Desquamation | Desquamation | Cells(#)-PMNs(#), | Cells(#)-PMNs(#), |
| Submucosa | Submucosa | Submucosa | Submucosa | Eosinophils(#) | Eosinophils(#) |
| Edema(mm) | Edema(mm) | Edema(mm) | Edema(mm) | Microgranulomas | Multinucleated-Giant |
| Cells(#)-PMNs(#), | Cells(#)-PMNs(#), | Cells(#)-PMNs(#), | Cells(#)-PMNs(#), | Cells(#)-PMNs(#), | Cells(#) |
| Eosinophils(#) | Eosinophils(#) | Eosinophils(#) | Eosinophils(#) | Eosinophils(#) | Blood Vessels |
| Granulomas | Granulomas | Granulomas | Granulomas | Multinucleated-Giant | Perivascular edema |
| Blood Vessels | Blood Vessels | Blood Vessels | Blood Vessels | Cells(#) | Perivascular cuffing |
| Perivascular edema | Perivascular edema | Perivascular edema | Perivascular edema | Blood Vessels | Cells(#)-PMNS(#), |
| Perivascular | Perivascular | Perivascular | Perivascular | Perivascular edema | Eosinophils(#) |
| cuffing | cuffing | cuffing | cuffing | Perivascular cuffing | |
| Cells(#)-PMNS(#), | Cells(#)-PMNS(#), | Cells(#)-PMNS(#), | Cells(#)-PMNS(#), | Cells(#)-PMNS(#) | |
| Eosinophils(#) | Eosinophils(#) | Eosinophils(#) | Eosinophils(#) | Eosinophils(#) | |

Each of the parameters evaluated were given an individual number score. The cumulative score was obtained using the individual scores and designated as no inflammation (O), mild inflammation (1-2), moderate inflammation (3-4), and severe inflammation (5-6). (mm=millimeter)

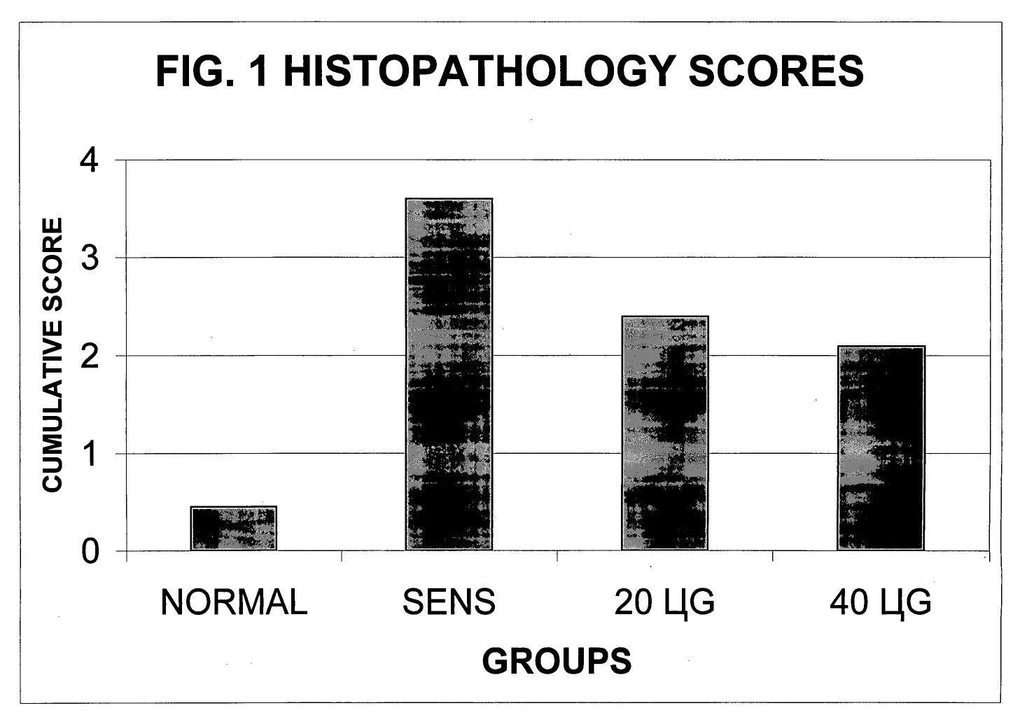

Composite test results for all tests are shown in FIG. 1, “Histopathology Score”. Significant reduction in total lung inflammation scores was noted with weekly treatments of 20 μg (p=0.046 and 40 μg (p=0.030) doses of triamcinolone encapsulated in sterically stabilized liposomes when compared to the untreated sensitized mice. The Figures refer to normal, unsensitized, untreated mice; “sens” refers to sensitized but untreated mice; the numbers “20 μg” and “40 μg” refer to groups of sensitized mice treated with these quantities of triamcinolone encapsulated in sterically stabilized liposomes; and, p refers to a probability value. Values of P that are less than 0.05 indicate significant values. Similar nomenclature is used with the other figures.

Eosinophil Peroxidase Activity (EPO) in BAL and Eosinophil Counts in Peripheral Blood and BAL

Eosinophil peroxidase activity in the BAL was assessed using a modified approach. Substrate solutions consisting of 0.1M Na Citrate, O-phenylenediamine and H₂O₂ (3%), at pH of 4.5, were mixed with BAL supernatants 1:1. The reaction mixture was incubated at 37° C. and the reaction was stopped by adding 4 N H₂SO₄. EPO activity was measured by spectrophotometric analysis at 490 nm. Horseradish peroxidase was used as a standard. The concentration of EPO activity was measured in nanograms per milliliter (ngm/ml).

The percentages of eosinophils from the peripheral blood smears were obtained by counting the number of eosinophils in 100 white blood cells under a high power field (100×). The smears are fixed and stained by Giemsa stain. Differential cell counts were determined from at least 200 leukocytes.

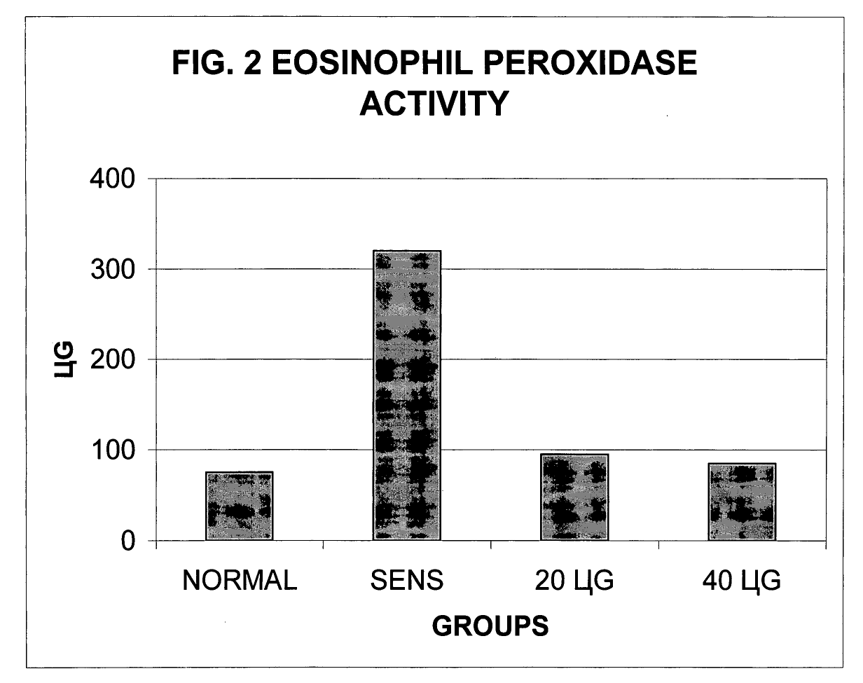

The composite test results for all tests are shown in FIG. 2, “Eosinophil peroxidase Activity (EPO).” Weekly treatments of 20 μg (p=0.0008 and 40 μg (p=0.0013) doses of triamcinolone encapsulated in sterically stabilized liposomes significantly decreased the EPO activity in the BAL when compared to the untreated sensitized mice and was comparable to normal untreated, unsensitized mice.

Total Serum IgE

Serum was separated from blood drawn at the time of sacrifice. Ninety-six well flat-bottom plates (Fisher Scientific, Pittsburgh, Pa.) were coated with 100 μl/well of 2 μg/ml rat anti-mouse IgE monoclonal antibody (BD PharMingen, San Diego, Calif.) and incubated overnight at 4° C. The plates were washed 3× with PBS plus Tween 20. The reaction was blocked with 1% bovine serum albumin (BSA) (Sigma Chemicals) in PBS and then washed 3× with PBS. Serum diluted at 1:50 with 1% BSA in PBS was pipetted at 100 μl/well, and incubated overnight at 4° C.

Purified mouse IgE (k isotype, small b allotype anti-TNP; BD PharMingen, San Diego, Calif.) was used as the standard for total IgE. The samples were washed 4× with PBS plus Tween 20. After washing, biotin-conjugated rat anti-mouse IgE (detection antibody, Southern Biotechnology, Birmingham, Ala.) was added to the wells and incubated for 1 hour. The samples were washed with PBS plus Tween 20 four times, then incubated with 100 μl/well (1:2000) streptavidin peroxidase (Sigma Chemicals, St. Louis, Mo.) for 1 hour. The samples were washed 4× with PBS plus Tween 20, developed using 100 μl sodium citrate buffer consisting of 0.1M Na citrate, 0.1M citric acid, 30 mg o-phenylendiamine, and 3% H₂O₂ (Sigma Chemicals, St. Louis, Mo.) at pH 4.0 for 15 minutes. The reaction was stopped by adding 50 μl of 6 N sulfuric acid. The samples were read at 490 nm by spectrophotometric analysis.

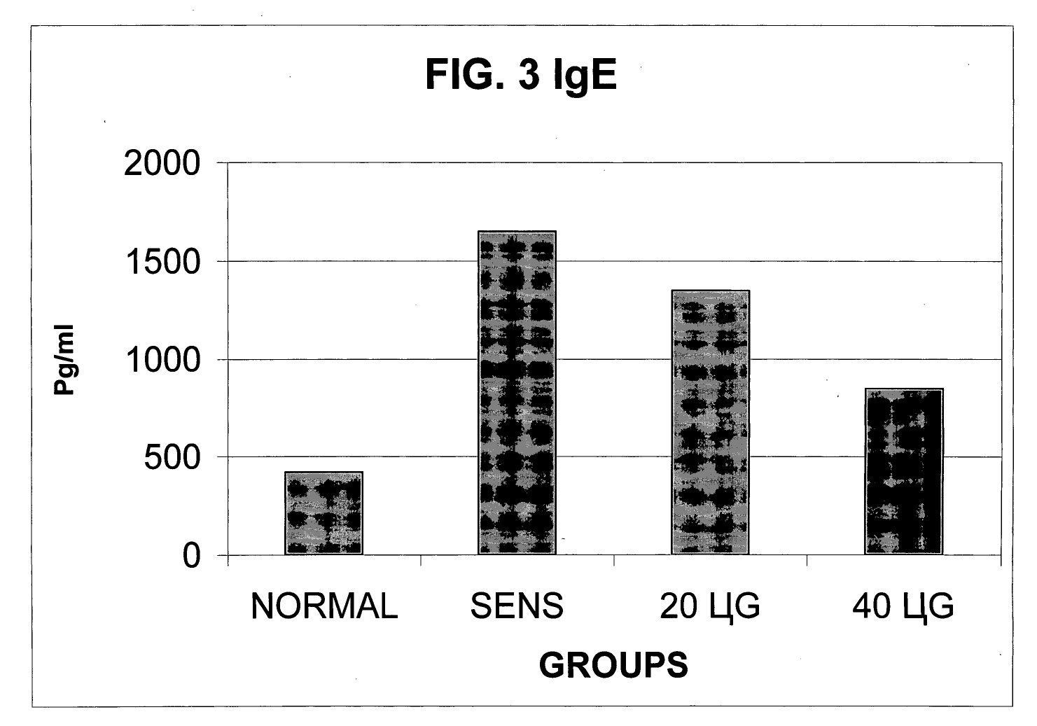

The composite test results for all tests are shown in FIG. 3, “IgE.” Treatments with 20 μg (p=0.148) and 40 μg (p=0.024) doses of weekly triamcinolone encapsulated in sterically stabilized liposomes significantly decreased the total serum IgE level. The 20 μg dose did not reduce the total serum IgE level as well as the 40 μg dose.

Airway Responsiveness to Methacholine

Pulmonary mechanics were studied using a modified protocol. Measurements to evaluate the effect of drug or drug-liposome therapy on airway responses to methacholine challenge were determined on spontaneously breathing mice that were tracheally intubated. The treatment groups were compared to sensitized, untreated mice and to healthy, normal mice undergoing the same procedures and receiving the same doses of methacholine. Methacholine challenges were determined every two weeks for 16 weeks on all experimental groups. As an antigen challenge and to demonstrate sensitization, an aerosolized dose of 6% ovalbumin was given to each animal 24 hours before the evaluation of the pulmonary mechanics.

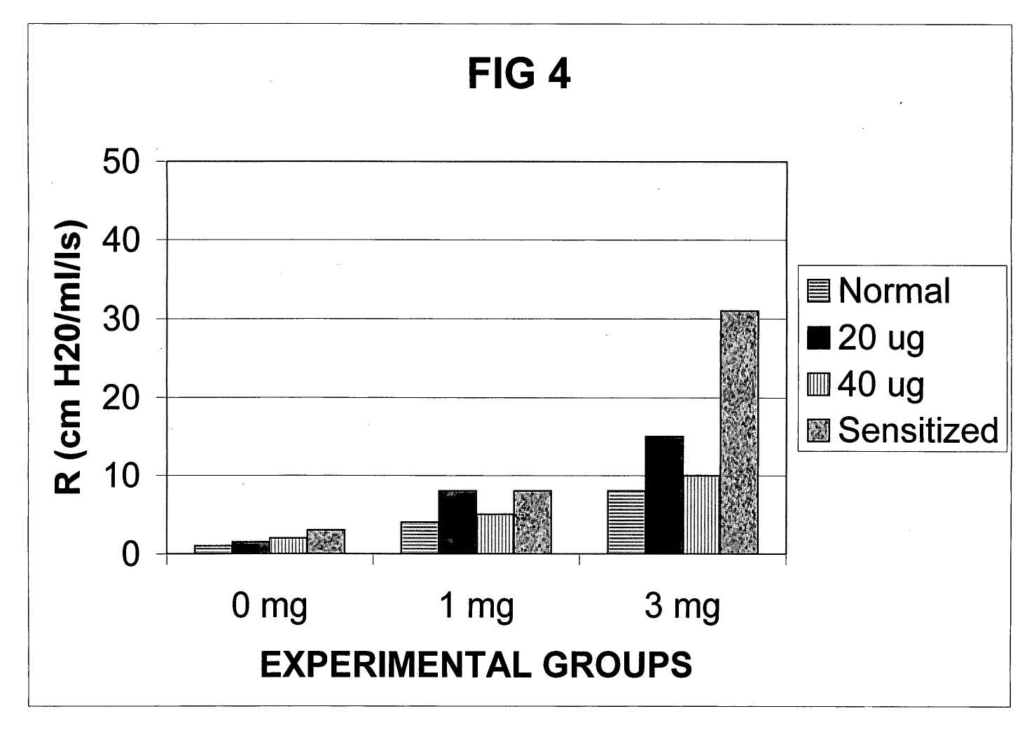

The composite test results for all tests for airway hyperresponsiveness to methacholine challenge are shown in FIG. 4 and shows significant decreases in both the 20 μg and 40 μg weekly treatment groups of triamcinolone encapsulated in sterically stabilized liposomes (p=0.001) when compared to the untreated sensitized mice

The animals were anesthetized with an intraperitoneal injection of a solution of ketamine and xylazine (40 mg/kg body weight for each drug). A 20 mg/kg body weight maintenance dose of pentobarbital sodium was given, before placement in the body plethysmograph. These doses were noted to maintain a steady level of anesthesia without causing significant respiratory depression in our preliminary studies.

A tracheotomy was done followed by the placement of a tracheostomy tube which was to be connected to a tube through the wall of body plethysmograph chamber, allowing the animal to breathe room air spontaneously. A saline-filled polyethylene tube with side holes was placed in the esophagus, and connected to a pressure transducer for measurements of flow, volume, and pressure. A screen pneumotachometer and a Valadyne differential pressure transducer will be used to measure flow in and out of the plethysmograph.

Signals from the pressure transducer and the pneumotachometer were processed using a Grass polygraph (model 7) recorder. The flow signal was integrated using a Grass polygraph integrator (model 7P10) to measure corresponding changes in pulmonary volume. Pressure, flow, and volume signal outputs were digitized and stored on a computer using an analog-to-digital data acquisition system (CODAS-Dataq Instruments, Inc., Akron, Ohio). The pressure and volume signals were also displayed to verify catheter placement and to monitor the animal during the experiment.

The digitalized data was analyzed for dynamic pulmonary compliance, pulmonary resistance, tidal volume, respiratory frequency and minute ventilation from about six to ten consecutive breaths at each recording event. Methacholine challenge was performed after baseline measurements were obtained. Methacholine was injected intraperitoneally, at 3-minute intervals, in successive cumulative doses of 30, 100, 300, 1000 and 3000 μg.

Data Analysis

Statistical analysis was performed using weekly, serial measurements from each group. Cumulative data for the sixteen-week period from each study group was presented as mean±standard error of the mean (SEM). One-way ANOVA with Tukey-Kramer multiple comparison data analysis was used for airway responses to methacholine challenge using SigmaStat Statistical Software (SPSS Science). EPO activity, EOS, IgE levels and histopathology scores were analyzed using the Student t test. P values <0.05 were considered to be statistically significant for all of the statistical comparisons.

Treatment Groups

Therapy was initiated on day 25, one day after the OVA sensitization was completed. Sensitized animals received nebulized treatments for four weeks as follows: (Group 1) 20 μg of triamcinolone encapsulated in sterically stabilized liposomes, once a week; (Group 2) 40 μg of triamcinolone encapsulated in sterically stabilized liposomes, once a week; (Group 3) buffer-loaded (empty) sterically stabilized liposomes, once a week. All treatment groups were compared to either 1) untreated sensitized animals or 2) untreated unsensitized (normal) mice.

The dose of triamcinolone was extrapolated from our previous studies dose responses with 5 μg and 50 μg of budesonide. 5 μg, 10 μg, 15 μg, 20 μg, or 50 μg of budesonide was administered via nebulization daily to a group of sensitized mice and the dose dependent effects on the inflammatory parameters were evaluated. These data were compared to either a group of untreated sensitized or unsensitized (normal) mice. A 20 μg dose of budesonide effectively decreased EPO activity in BAL, EOS, and inflammation on histopathological examination of the lung tissues, along with other inflammatory parameters studied, without evidence of toxicity to the spleen, liver, bone marrow, skin or the gastrointestinal tract. We could not conduct a similar dose response study with triamcinolone since at present there are no means to nebulize free triamcinolone. We also used the 40 μg dose, although 20 μg may have shown efficacy, because a higher dose of triamcinolone is used to treat human asthma.

Experiments were performed to evaluate the progression of inflammation and bronchoconstriction in the asthma model. Each treatment group consisted of 20 mice followed for a four-week period. Five animals from each treatment and control groups were sacrificed by overdose of methoxyflurane by inhalation 24 hours after the first treatments were given, and then every week for 4 weeks. At each time point serial measurements of EPO in BAL, eosinophil counts in BAL and peripheral blood, total serum IgE level, and histopathological examination of the lung tissues were obtained. The assays were performed as described in the methods section.

In addition, before embarking upon testing the effect of triamcinolone encapsulated in liposomes therapy in humans, especially in children, it is preferable to test its validity and safety in animals wherein asthma is induced experimentally. While there are several animal models to choose from, ranging from mice, guinea pig, monkey, and dog, etc., we chose to use the mouse, specifically C57B1/6 mice in these studies. Our decision to use the C57/B1/6 mice is dictated by several advantages including the ease of sensitization to ovalbumin and the possibility of future genetic studies. There are models of C57B1/6 mice commercially available in which genetic manipulation has been shown to impact the progression of asthma i.e. IL-4 or IL-13 knockout mice.

Our preliminary data have shown that this method of sensitization produces significant elevations in eosinophil peroxidase (EPO) activity, peripheral blood eosinophil count (EOS), serum IgE levels, along with significant lung inflammation on histopathological examination and increased airway hyperresponsiveness to methacholine challenge, by day 24. We have also noted continued lung inflammation, elevations in EPO activity, EOS, serum IgE levels, and airway hyperresponsiveness upon rechallenge with ovalbumin at 2 months, 4 months and 5 months after sensitization was completed.

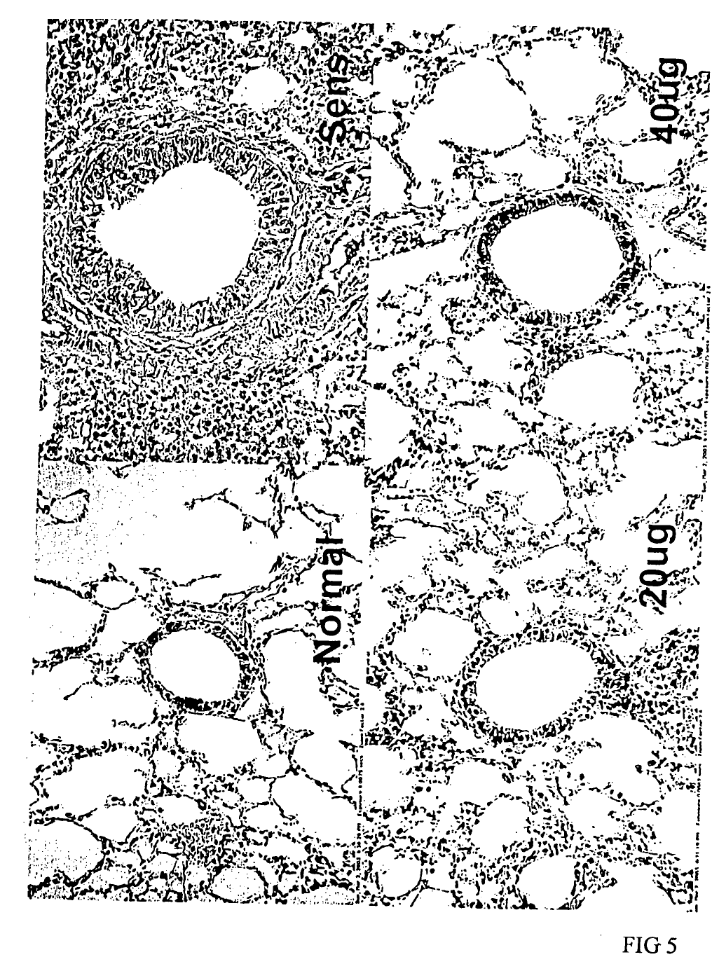

Examples of lung tissues from the four experimental groups are shown below in FIG. 5 (A=normal; B=sensitized, untreated; C and D=20 μg doses of weekly triamcinolone encapsulated in sterically stabilized liposomes, respectively). Significant reduction in total lung inflammation score (FIG. 3) was noted with weekly treatments of 20 μg (p=0.046 and 40 μg (p=0.030) doses of triamcinolone encapsulated in sterically stabilized liposomes when compared to the untreated sensitized mice

Results

Legend: Normal=normal, untreated, unsensitized; Sens=sensitized, untreated; 20 μg=20 μg of nebulized triamcinolone in sterically stabilized liposomes; 40 μg=40 μg of nebulized triamcinolone in sterically stabilized liposomes (*) indicates significance at p<0.05.

The test results clearly show the surprising effectiveness of triamcinolone treatment as described above for treatment of lung inflammation and airway hyperresponsiveness as well as the other test results shown herein.

The data have shown that 20 μg or 40 μg of triamcinolone encapsulated in sterically stabilized liposomes and given once a week reduced inflammation as effectively as budesonide which was given once a day. Weekly treatments with free budesonide, budesonide encapsulated in conventional liposomes or empty sterically stabilized or conventional liposomes did not decrease inflammation as well as triamcinolone encapsulated in sterically stabilized liposomes.

The results of the preliminary studies have shown that triamcinolone encapsulated in sterically stabilized liposomes provides an effective means to decrease lung inflammation in experimental asthma. Both 20 μg and 40 μg was found to significantly decrease lung inflammation. Levels of immunological markers implicated in the progression of asthma including bronchio-alveolar lavage (BAL) eosinophil peroxidase activity (EPO) and peripheral blood eosinophil count (EOS) have decreased significantly with our approach. Eosinophil peroxidase, one of the major eosinophil cytotoxins, has been identified as an important mediator of airway inflammation. It has ribonuclease activity and is a potent helminthotoxin and neurotoxin.

Eosinophils have a primary role in the inflammatory phase of asthma, as they secrete cytotoxins that directly damage lung mucosa and epithelium. In preliminary experiments, peripheral blood eosinophil counts were significantly decreased. This is consistent with previous reports of a decrease in pro-inflammatory cytokine production with inhaled steroids, which in turn inhibit bone marrow production or release of eosinophils. Consistent with these previous studies, decreased levels of IL-4 and IL-5 in BAL and splenocyte culture supernatants (performed by ELISA, R&D System, Minneapolis, Minn.)) in the weekly triamcinolone encapsulated in sterically stabilized liposomes treatment groups were noted.

The histopathological changes in the lung tissue were evaluated to assess the degree of inflammation, including epithelial hyperplasia, basement membrane thickening and infiltration of inflammatory cells. There was a significant decrease in lung inflammation with weekly therapy with the triamcinolone encapsulated in sterically stabilized liposomes.

It has been found that weekly therapy with triamcinolone encapsulated in sterically stabilized liposomes significantly decreased airway hyperresponsiveness to methacholine challenge. Preliminary studies did not reveal any toxicity to the animals with weekly treatment of triamcinolone encapsulated in sterically stabilized liposomes.

This unique drug delivery system may provide a valuable alternative to daily corticosteroid therapy with a potential to reduce toxicity and improve compliance for inhaled steroid therapy in asthma. The prevalence of asthma has been increasing and affects a significant portion of the population. The cost to treat asthma has also been rising at an alarming rate. Thus it is imperative to find efficient mechanisms to lessen both the morbidity and the financial burden of this chronic disease.

While the present invention has been described by reference to certain of its preferred embodiments, it is pointed out that the embodiments described are illustrative rather than limiting in nature and that many variations and modifications are possible within the scope of the present invention. Many such variations and modifications may be considered obvious and desirable by those skilled in the art based upon a review of the foregoing description of preferred embodiments. These variations include but are not limited to different methods of liposome preparation, such as hydration of dried lipids followed by extrusion, reverse phase evaporation with or without extrusion, sonication, detergent dialysis and injection from an ethanolic solution.

What is claimed is:

1. A composition comprising a sterically stabilized liposome in combination with triamcinolone for aerosol administration, the sterically stabilized liposome being compatible with a respiratory tract of a mammal and effective to extend the effective life of the triamcinolone in the respiratory tract by a time equal to at least twice the effective life of the triamcinolone alone.

2. The composition of claim 1 wherein the time is equal to at least three times the effective life of the drug alone.

3. The composition of claim 1 wherein the carrier comprises at least one of phosphatidylcholine, phosphatidylglycerol and poly(ethylene) glycol.

4. The composition of claim 5 wherein the poly(ethylene glycol) has a molecular weight from about 500 to about 5,000 daltons.

5. The composition of claim 1 wherein at least one of phosphatidylcholine, phosphatidylglycerol, and poly(ethylene glycol) that is attached covalently to a lipid such as phosphatidylethanolamine, have acyl chains containing from about 8 to about 18 carbon atoms.

6. The composition of claim 5 wherein the acyl groups comprise at least one of distearoyl, stearoyl oleoyl, stearoyl palmitoyl, dipalmitoyl, dioleoyl, palmitoyl oleoyl and dipalmitoleoyl.

7. The composition of claim 1 wherein the sterically stabilized liposome comprises at least one of poly(ethylene glycol)-conjugated lipids, phosphatidylinositol, dipalmitoylphosphatidyl-polyglycerol, lipid-conjugated polyoxyethylene, lipid-conjugated polysorbate, or lipids conjugated to other hydrophilic steric coating molecules safe for in vivo use, the sterically stabilized liposome being effective to extend the effective lifetime of triamcinolone in the respiratory tract of a mammal.

8. The composition of claim 1 wherein the sterically stabilized liposome is phosphatidylcholine, phosphatidylglycerol, poly(ethylene glycol)-distearyolphosphatidyldiethanolamine, with or without cholesterol.

9. The composition of claim 1 wherein the sterically stabilized liposome comprises at least one of egg-derived or soybean-derived phosphatidylcholine and phosphatidylglycerol.

10. The composition of claim 1 wherein the phosphatidylcholine is present in an amount equal to from about 0 to about 99 weight percent.

11. The composition of claim 10 wherein the sterically stabilized liposome comprises from about 0 to about 99 weight percent phosphatidylglycerol.

12. The composition of claim 1 wherein the sterically stabilized liposome contains at least one of phosphatidylcholine, phosphatidylglycerol or poly(ethylene glycol)-derivatized lipid having acyl chains containing from about 10 to about 40 carbon atoms.

13. The composition of claim 12 wherein the acyl groups comprise at least one of distearoyl, stearoyl oleoyl, stearoyl palmitoyl, dipalmitoyl, dioleoyl, palmitoyl oleoyl and dipalmitoleoyl.

14. The composition of claim 1 wherein the sterically stabilized liposome comprises phosphatidylethanolamine and a protonatable lipid.

15. The composition of claim 14 wherein the protonatable lipid is selected from the group comprising cholesterol hemisuccinate, diacylsuccinylglycerol and oleic acid.

16. A method for treating a respiratory tract of a mammal by aerosol administration of an effective amount of a composition comprising a sterically stabilized liposome in combination with triamcinolone, the sterically stabilized liposome being compatible with the respiratory tract of a mammal and effective to extend the effective life of triamcinolone in the respiratory tract by a time equal to at least twice the effective life of triamcinolone alone.

17. The method of claim 16 wherein the sterically stabilized liposome comprises at least one of phosphatidylcholine and phosphatidylglycerol.

18. The method of claim 17 wherein the phosphatidylcholine is present in an amount equal to from about 50 to about 100 weight percent.

19. The carrier of claim 17 wherein the sterically stabilized liposome comprises from about 0 to about 50 weight percent phosphatidylglycerol.

20. The method of claim 17 wherein the sterically stabilized liposome further comprises poly(ethylene glycol) attached to a lipid.

21. The method of claim 16 wherein the sterically stabilized liposome comprises at least one of phosphatidylcholine, phosphatidylglycerol, and poly(ethylene glycol) attached to a lipid such as phosphatidylethanolamine, having acyl chains containing from about 8 to about 18 carbon atoms.

22. The method of claim 21 wherein the acyl groups comprise at least one of distearoyl, stearoyl oleoyl, stearoyl palmitoyl, dipalmitoyl, dioleoyl, palmitoyl oleoyl and dipalmitoleoyl.

23. The method of claim 16 wherein the sterically stabilized liposome comprises at least one of poly(ethylene glycol)-conjugated lipids, phosphatidylinositol, dipalmitoylphosphatidylpolyglycerol, lipid-conjugated polyoxyethylene, lipid-conjugated polysorbate, or lipids conjugated to other hydrophilic steric coating molecules safe for in vivo use, the sterically stabilized liposome being effective to extend the effective lifetime of a drug in the respiratory tract of a mammal.

24. The method of claim 16 wherein the sterically stabilized liposome is phosphatidylcholine, phosphatidylglycerol, poly(ethylene glycol)-distearoylphosphatidylethanolamine, with or without cholesterol.

25. The method of claim 16 wherein the sterically stabilized liposome comprises at least one egg-derived or soybean derived phosphatidylglycerol or phosphatidylglycerol.

26. The method of claim 16 wherein the liposome comprises of phosphatidylcholine, phosphatidylglycerol, and poly(ethylene glycol), distearoylphosphatidylethanolamine and triamcinolone at a mole ratio of 8:2:0.5:2, with or without 3 parts cholesterol.

27. The method of claims 1 and 16 wherein the liposomes are prepared by hydration of a dried film followed by extrusion through polycarbonate membranes of a pore diameter ranging from 2 to 0.05 micrometers.
